(19) United States
(12) Plant Patent Application Publication
(10) Pub. No.: US 2015/0245551 P1
(43) Pub. Date: Aug. 27, 2015

(54) Pear tree named 'PremP009'

(50) ;

(71) Applicant: Allan G. White, Havelock North (NZ)

(72) Inventor: Allan G. White, Havelock North (NZ)

(21) Appl. No.: 14/544,845

(22) Filed: Feb. 24, 2015

Related U.S. Application Data
(60) Provisional application No. 61/966,638, filed on Feb. 26, 2014.

(51) Int. Cl. A01H 5/00

(52) U.S. Cl. PLT/176

(57) ABSTRACT

A new and distinct pear tree named ‘PremP009’ is disclosed. The new pear is notable for its attractive appearance, superb texture, improved flavor and long shelf life.

### LATIN NAME OF THE GENUS AND SPECIES OF THE PLANT CLAIMED

Pyrus communis×Pyrus pyrifolia×Pyrus bretschneideri

### VARIETY DENOMINATION

‘PremP009’

### BRIEF DESCRIPTION OF THE PHOTOGRAPHS

FIG. 1 shows fruit of the ‘PremP009’ variety; and

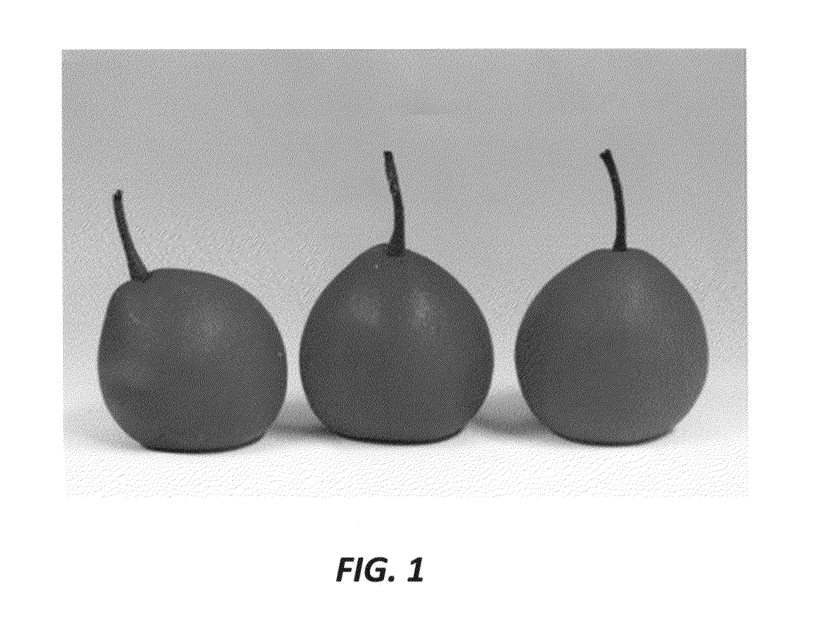

FIG. 2 shows fruit and leaves of the ‘PremP009’ variety.

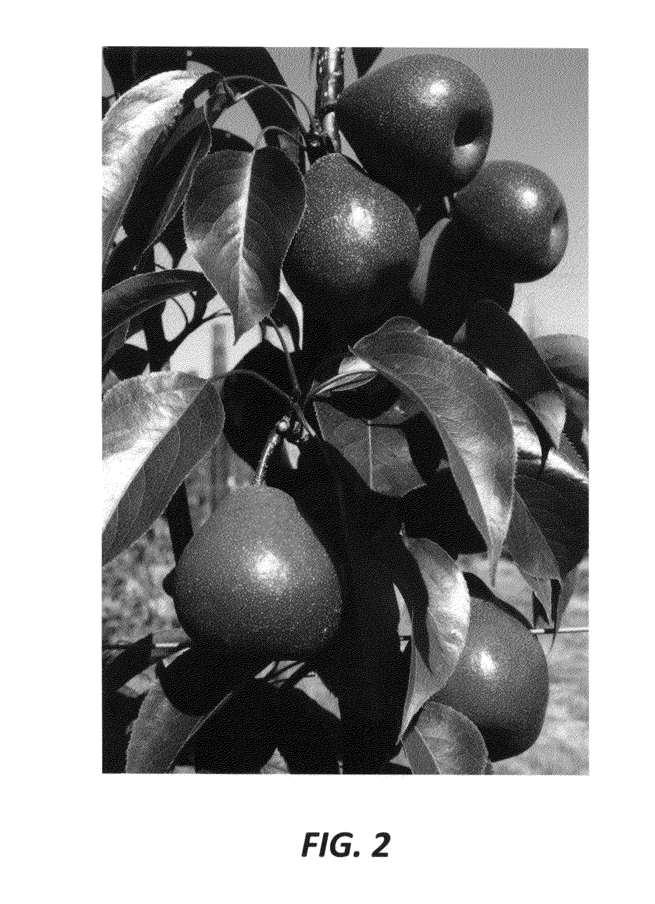

### BRIEF DESCRIPTION OF THE VARIETY

The new variety of pear tree ‘PremP009’ was created in the course of a planned breeding program carried out in Havelock North, New Zealand. ‘PremP009’ originated as a result of a controlled cross of ‘P128R068T003’ (seed parent, not patented) and ‘P204R135T058’ (pollen parent, not patented). ‘PremP009’ was selected in 2005 as a single plant from among a population of seedlings derived from the parents, and was selected for its fruit, which exhibits an attractive intense dark red skin color and unique appearance, superb texture and flavor, and long shelf life. ‘PremP009’ has a red skin color, which differs from the yellow skin color of its parent variety ‘P128R068T003’. ‘PremP009’ also has a medium to long period of postharvest storage compared to the short period of storage available to its pollen parent, ‘P204R135T058’. The following table compares ‘PremP009’ to a similar variety, ‘PremP109’ (pending U.S. Plant patent application Ser. No. 13/374,755).

| Characteristics in which | | |
|---|---|---|
| ‘PremP009’ differs from | | |
| ‘PremP109’ | ‘PremP109’ | ‘PremP009’ |
| Skin Color | Mid Red | Bright Mid to Dark Red |
| Harvest Maturity | Mid to Late Season | Mid to Late Season |
| Amount of Overcolor | Medium | High |
| Shape | Round | Pyriform |
| Postharvest Storage | 10 Weeks | 6-10 Weeks |
| Flavor Profile | European and | European and |
| | Nashi flavor | Nashi flavor |
| Flowering Time | Mid to Late Season | Late Season |

Asexual propagation by budding was first carried out in Havelock North, New Zealand. The variety has since been observed over a number of asexually propagated generations, and has been found to remain true to type.

### DETAILED BOTANICAL DESCRIPTION OF THE VARIETY

The following detailed botanical description is based on observations made during the 2013 growing season at Havelock North, New Zealand. All colors are described according to The Royal Horticultural Society Colour Chart (2001). It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Tree: Vigor — Moderate; Habit — Upright; Size — Height 3.5 m, spread 2.75 m; Trunk — Diameter at 5 cm above graft union 96 mm; Bark texture — smooth with raised lenticels; Bark color — Brown 200B; Lenticel color — Grey 201B.
Branch: Size — Length 1.75 M; Diameter 23 mm at 1.37 m above graft union; Branch color — 200B; Lenticel density — 2-3 per square cm; Lenticel size — Length 2 mm, Height 5 mm.
Dormant one-year-old shoot: Size — diameter 3-5 mm; Internode length 30-35 mm; Color — 200A; Lenticels — quantity 2-3 per square cm, color 199C, diameter 2 mm
Flowers: Time of flowering — very late; Bloom period — First bloom October 8, full bloom October 23 in Hawkes Bay, New Zealand.
Leaf: Attitude in relation to shoot — upward; Blade margin — serrated; Size — length 12.2 cm, width 8.1 cm; Color — upper surface 146D; Color of lower surface — 145C; Apex with petiole — 2.8 cm; Attitude in relation to shoot — 45°.
Fruit: Size — Height 70 mm, Width 60 mm; Shape — Pyriform; Quantity per cluster 3-5; Depth of eye basin — 10 mm; Width of eye basin — 17 mm; Length of stalk — 28 mm; Thickness of stalk 3 mm; Size of lenticels — small 0.25 mm; Greasiness of skin — slight; Color of skin Grey Purple Group 187 B; Intensity of overcolor — medium at harvest, bright at post storage; Flesh texture — Fine, crisp, juicy; Juiciness — Moderate; Brix 13-14°; Flesh color — White 155D; Stem color — Brown 200A; Seeds — Quantity per fruit 8-12; Seed color — Brown 200B.
Harvest: Amount of fruit produced per tree per harvest — 56 at 5^{th} leaf; Harvest date range — February 25^{th} at Hawkes Bay, NZ.
Disease resistance/susceptibility: Resistant to pear scab; Moderately susceptible to fire blight.
Storage: Excellent storage performance of at least 10 weeks and up to 16 weeks and 14 days ambient shelf life.
Use: Fresh Market.

I claim:

1. A new and distinct pear tree substantially as shown and described herein.
